(19) United States
(12) Patent Application Publication
(10) Pub. No.: US 2023/0038423 A1
(43) Pub. Date: Feb. 9, 2023

(54) ORAL CANNABINOID FORMULATIONS

(71) Applicant: GW Research Limited, Cambridge (GB)

(72) Inventors: Alan SILCOCK, Cambridge (GB); Jitinder WILKHU, Cambridge (GB); Tu Ching THAI, Cambridge (GB)

(21) Appl. No.: 17/786,949

(22) PCT Filed: Dec. 18, 2020
(86) PCT No.: PCT/GB2020/053282

§ 371 Date: Jun. 17, 2022

(30) Foreign Application Priority Data
Dec. 19, 2019 (GB) 1918846.5

(51) Int. Cl. A61K 31/05; A61K 9/08; A61K 9/00; A61K 31/352; A61K 31/192; A61K 47/44; A61K 47/10; A61K 47/14

(52) U.S. Cl. CPC A61K 31/05; A61K 9/08; A61K 9/0053; A61K 31/352; A61K 31/192; A61K 47/44; A61K 47/10; A61K 47/14

(57) ABSTRACT

The present invention relates to a cannabinoid containing oral solution. Preferably the cannabinoid is cannabidiol (CBD), cannabidivarin (CBDV) or cannabidiol-C4 (CBD-C4). More preferably the CBD, CBDV or CBD-C4 is present at a concentration of between 25 and 75 mg/ml. More preferably still the CBD, CBDV or CBD-C4 is present at a concentration of 50 mg/ml. In a further embodiment the oral solution is formulated with one or more edible oils. Preferably the edible oil is sesame oil. It is a preferred embodiment of the invention that the oral formulation comprises a low concentration of ethanol.

The present invention relates to a cannabinoid containing oral solution. Preferably the cannabinoid is cannabidiol (CBD), cannabidivarin (CBDV) or cannabidiol-C4 (CBD-C4). More preferably the CBD, CBDV or CBD-C4 is present at a concentration of between 25 and 75 mg/ml. More preferably still the CBD, CBDV or CBD-C4 is present at a concentration of 50 mg/ml.

In a further embodiment the oral solution is formulated with one or more edible oils. Preferably the edible oil is sesame oil. It is a preferred embodiment of the invention that the oral formulation comprises a low concentration of ethanol.

### BACKGROUND TO THE INVENTION

The use of cannabinoids in medicine has necessitated finding more effective ways of drug delivery. This is in part due to factors such as, poor aqueous solubility, limited bioavailability, and cannabinoid instability, but the use of cannabinoids at relatively high doses (in daily amounts of up to 2000 mg) and/or in challenging patient groups, e.g. young children, and/or for particular indications, can create additional challenges.

There are currently four commercially available cannabinoid formulations on the market.

Dronabinol (Marinol®) is a synthetic tetrahydrocannabinol (THC) which is delivered orally, in sesame oil as capsules.

Nabilone (Cesamet®) is a synthetic cannabinoid and an analog of THC and is delivered orally in capsules with povidone and corn starch.

Nabiximols (Sativex®) is a natural extract of cannabinoids containing defined amounts of THC and Cannabidiol (CBD) and is delivered as a liquid, by way of an oromucosal spray.

Epidiolex® or Epidyolex® is an oral solution containing 100 mg/ml which is approved for the treatment of seizures associated with Dravet syndrome and Lennox-Gastaut syndrome. The CBD is formulated in sesame seed oil and further comprises the sweetener sucralose, strawberry flavouring and up to 10% v/v ethanol.

Whilst there is no clear FDA guidance for maximum allowable ethanol concentration in prescription medicines, an article (Ethanol in Liquid Preparations Intended for Children, Paediatrics: Official Journal of The American Academy of Paediatrics, 1984: 73:405), recommends that a Blood Alcohol Concentration (BAC) of 0.25 g/L (250 mg/L) should not be exceeded following a single dose of alcohol containing medications.

WO 2015/184127 (Insys) discloses a number of different oral formulations including: an alcohol-free formulation in which the cannabinoid is formulated in a mix of polyethylene glycol and propylene glycol, optionally with water, a formulation containing alcohol and a formulation containing lipids. In each of the formulations disclosed, the cannabinoid is a synthetically produced (as opposed to a naturally extracted) cannabidiol. CBD is present in the formulations between 1 and 35%.

According to European Medicine Agency draft guideline (EMA/CHMP/507988/2013), for 2 to 6 years old children, a theoretical limit for Blood Alcohol Concentration (BAC) following single administration of a formulation containing alcohol is not more than 0.01 g/L (10 mg/L) and ethanol intake should be not more than 6 mg/kg/day.

For paediatric products aimed at younger children, it is desirable to have low or no ethanol formulations, preferably dispensed as syrup, as younger children find it difficult to swallow capsules. They also favour sweet, flavoured products, particularly where the taste of cannabinoid requires masking.

Pharmaceutically acceptable sweeteners and flavouring agents are generally polar in nature, and thus unlike the cannabinoids which are highly lipophilic, they require a polar solvent to dissolve them.

Oral delivery of cannabinoids generally results in poor bioavailability, for example Epidiolex which is provided as a 100 mg/ml oral solution in sesame oil has a mean Cₘₐₓ of 189 ng/ml and a mean AUC₀₋ₜ of 995 ng.h/ml. Young children require an oral formulation as they are unable to swallow a solid dosage form, however due to the poor bioavailability of a formulation such as Epidiolex large volumes of the medicine are required to be given. This presents a problem due to the gastrointestinal problems associated with a large intake of edible oils such as sesame oil.

An object of the present invention is to develop a lipid based oral formulation which is able to provide a high bioavailability.

Surprisingly the applicant has found that by decreasing the concentration of the cannabinoid in the oral formulation there results in an increase in the bioavailability. This was unexpected and leads to a beneficial effect.

### BRIEF SUMMARY OF THE DISCLOSURE

In accordance with a first aspect of the present invention there is provided a cannabinoid containing oral solution which comprises: a cannabinoid and a lipid solvent, characterised in that the cannabinoid is present in a concentration of from 25 to 75 mg/ml.

In one embodiment the Cₘₐₓ produced in a human is greater than 250 ng/ml.

In a further embodiment the AUC₀₋ₜ produced in a human is greater than 1250 ng.h/ml.

Preferably the cannabinoid is selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV); tetrahydrocannabivarinic acid (THCVA); cannabidiol-C1 (CBD-C1); cannabidiol-C4 (CBD-C4); and cannabidiol-C6 (CBD-C6).

More preferably the cannabinoid is cannabidiol (CBD).

In a further embodiment the cannabinoid is present at a concentration of approximately 50 mg/ml.

Preferably the lipid solvent is an edible oil. More preferably the edible oil is selected from: coconut oil; corn oil; cottonseed oil; hemp oil; olive oil; palm oil; peanut oil; rapeseed/canola oil; safflower oil; sesame oil; soybean oil; short chain triglyceride; medium chain triglyceride; long chain triglyceride and sunflower oil.

Preferably the cannabinoid is cannabidiol (CBD) and the edible oil is sesame oil.

Preferably the cannabinoid is cannabidiol (CBD) and the edible oil is soybean oil.

Preferably the cannabinoid is cannabidiol (CBD) and the edible oil is olive oil.

Preferably the cannabinoid is cannabidiol (CBD) and the edible oil is medium chain triglyceride.

Preferably the cannabinoid is cannabidivarin (CBDV) and the edible oil is sesame oil.

Preferably the cannabinoid is cannabidiol-C4 (CBD-C4) and the edible oil is sesame oil.

Preferably the cannabinoid is cannabidiol-C4 (CBD-C4) and the edible oil is soybean oil.

Preferably the cannabinoid is cannabidiol-C4 (CBD-C4) and the edible oil is olive oil.

Preferably the cannabinoid is cannabidiol-C4 (CBD-C4) and the edible oil is medium chain triglyceride.

Preferably the formulation further comprises ethanol. Preferably the ethanol is present at less than 10% w/v. More preferably the ethanol is present at less than 1% w/v.

Preferably the cannabinoid containing oral solution is for use in the treatment of a disease or disorder selected from the group consisting of: epilepsy and syndromes associated therewith, Dravet Syndrome, Lennox Gastaut Syndrome, myocolonic seizures, juvenile mycolonic epilepsy, refractory epilepsy, schizophrenia, juvenile spasms, West syndrome, infantile spasms, refractory infantile spasms, tuberous sclerosis complex, brain tumors, neuropathic pain, cannabis use disorder, post-traumatic stress disorder, anxiety, early psychosis, Alzheimer's Disease, and autism.

### BRIEF DESCRIPTION OF THE DRAWINGS

Embodiments of the invention are further described hereinafter with reference to the accompanying drawings, in which:

FIG. 1 shows the PK profile of CBD after testing with four different formulations;

FIG. 2 shows the PK profile of 7-OH CBD after testing with four different formulations; and

FIG. 3 shows the PK profile of 7-COOH CBD after testing with four different formulations.

FIG. 4 shows the effect of varying dose concentration of CBD formulated in different oils.

FIG. 5 shows the effect of varying dose concentration of CBDV.

FIG. 6 shows the effect of varying dose concentration of CBD-C4 formulated in different oils.

### DETAILED DESCRIPTION

Oral delivery of cannabinoids generally results in poor bioavailability, for example Epidiolex which is provided as a 100 mg/ml oral solution in sesame oil has a Cₘₐₓ of 189 ng/ml and an AUC₀₋ₜ of 995 ng.h/ml. Young children require an oral formulation as they are unable to swallow a solid dosage form, however due to the poor bioavailability of a formulation such as Epidiolex large volumes of the medicine are required to be given. This presents a problem due to the gastrointestinal problems associated with a large intake of edible oils such as sesame oil.

An object of the present invention is to develop a lipid based oral formulation which is able to provide a high bioavailability.

Surprisingly the applicant has found that by decreasing the concentration of the cannabinoid in the oral formulation there results in an increase in the bioavailability. This was unexpected and leads to a beneficial effect.

The Examples that follow describe the development of the claimed formulations which provide increased bioavailability.

### Pharmacokinetic Parameters

| | |
|---|---|
| Cₘₐₓ | Maximum observed concentration. |
| Tₘₐₓ | Time of maximum observed concentration. |
| AUC₀₋ₜ or | Area under the plasma concentration-time curve from hour 0 to the last |
| AUCₗₐₛₜ | measurable concentration, estimated by the linear trapezoidal rule. |
| AUC_{0-inf} | Area under the plasma concentration-time curve from time 0 to infinity, |
| | calculated as |
| | AUC_{0-inf} = AUC ₀₋ₜ + Cₜ/λ_{Z}, where Cₜ is the last observed quantifiable |
| | concentration and A_{z} is the elimination rate constant. |
| t_{1/2} | Elimination half-life, determined by In(2)/λ_{Z} |
| CL/F | Apparent total clearance of the drug from plasma after oral administration |
| V_{z}/F | Apparent volume of distribution during terminal phase after non-intravenous |
| | administration |
| M:P | Metabolite to parent ratio, calculated as: |
| | [Cₘₐₓ/D and AUC₀₋₂₄/D 7-OH-CBD]/[Cₘₐₓ/D and AUC₀₋₂₄/D Parent] |
| | [Cₘₐₓ/D and AUC₀₋₂₄/D 7-COOH-CBD]/[Cₘₐₓ/D and AUC₀₋₂₄/D Parent] |

### Example 1— Pharmacokinetic Testing of Formulations

The pharmacokinetic properties of various oral formulations comprising cannabidiol (CBD) were tested.

The composition of these formulations are described in Table 1 below.

TABLE 1
Composition of formulations

| | CBD | | |
|---|---|---|---|
| Formulation | concentration | | Ethanol |
| No. | (mg/ml) | Edible oil | (% w/v) |
| 1 | 100 | Sesame oil | 7.9 |
| 2 | 50 | Sesame oil | 0.79 |
| 3 | 100 | Sesame oil | 0.79 |
| 4 | 150 | Sesame oil | 0.79 |

Formulations additionally comprised sweetener and flavouring.

These formulations were tested in healthy volunteers as per the following protocol.

### Protocol Details

Subjects were randomized to 1 of 4 treatment sequences in which they were administered single oral doses of each of the CBD formulations in Treatment Periods 1 to 4:

Test Treatment 1: A single oral dose of 750 mg CBD (100 mg/ml CBD) under fasted conditions with a total volume of 7.5 ml.

Test Treatment 2: A single oral dose of 750 mg CBD reduced ethanol formulation (50 mg/ml CBD) under fasted conditions with a total volume of 15 ml.

Test Treatment 3: A single oral dose of 750 mg CBD reduced ethanol formulation (100 mg/ml CBD) under fasted conditions with a total volume of 7.5 ml.

Test Treatment 4: A single oral dose of 750 mg CBD reduced ethanol formulation (150 mg/ml CBD) under fasted conditions with a total volume of 5 ml.

Pharmacokinetic properties were tested via blood sampling at the following times: 0, 0.5, 1, 1.5, 2, 2.5, 3, 4, 5, 6, 8, 10, 12 and 24 hours post-dose.

### Results

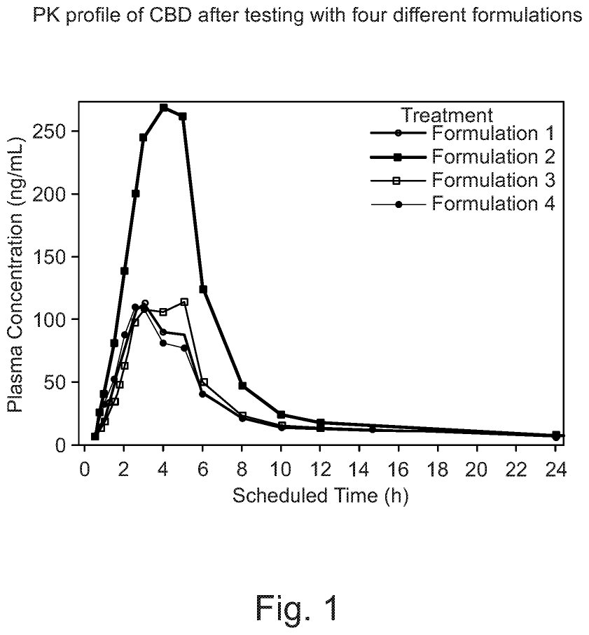

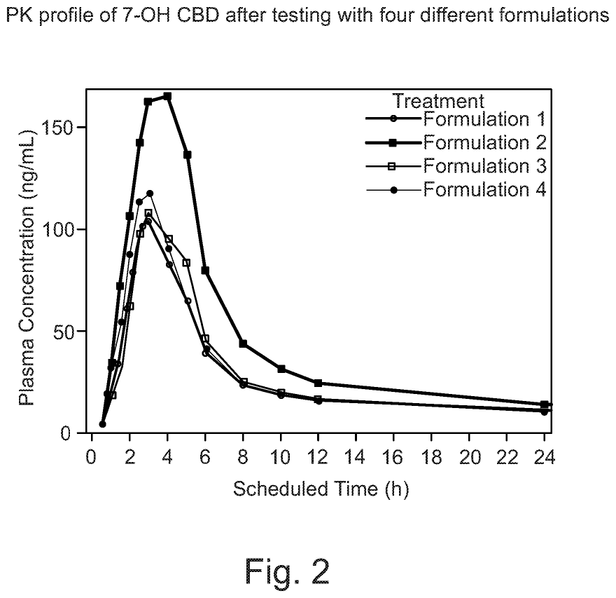

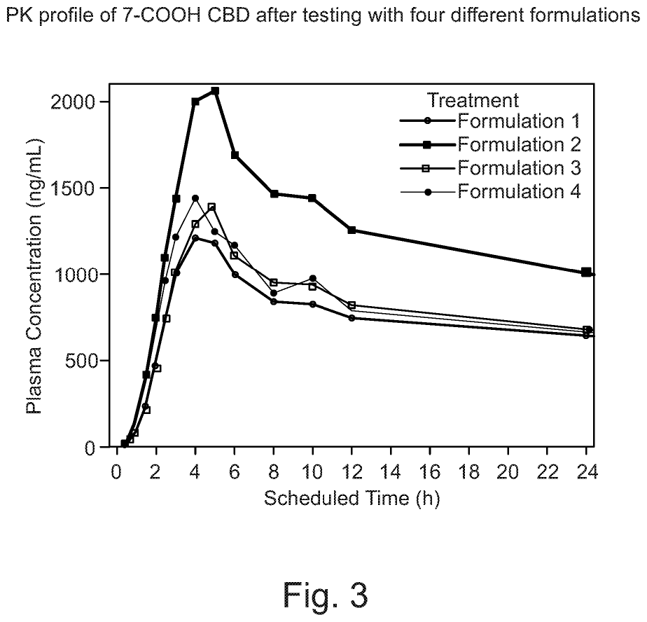

FIGS. 1 to 3 detail the PK profiles of CBD and its two main metabolites 7-OH CBD and 7-COOH CBD for the four different formulations tested.

As can be seen in FIG. 1, Formulation 2 produced a dramatic increase in the plasma concentration of CBD when compared to the other formulations. This was unexpected as this was the lower concentration of CBD at 50 mg/ml.

As would be expected, Formulation 2 also produced an increased concentration of the two metabolites 7-OH CBD and 7-COOH CBD when compared to the other formulations.

Tables 2 to 4 below summarise these data, where OS is Formulation 1; 50 mg/ml is Formulation 2; 100 mg/ml is Formulation 3 and 150 mg/ml is Formulation 4.

TABLE 2
Summary of PK results for CBD

| | | Cmax | Tmax | AUC₀₋ₜ | AUC_{0-∞} | t1/2 | CL/F | Vz/F |
|---|---|---|---|---|---|---|---|---|
| CBD | | (ng/mL) | (h) | (ng · h/mL) | (ng · h/mL) | (h) | (L/h) | (L) |
| OS | Mean | 189 | 3.14 | 995 | 1041 | 29.0 | 910 | 32788 |
| | SD | 126 | 1.20 | 472 | 484 | 13.1 | 500 | 13770 |
| | CV % | 66.5 | 38.2 | 47.5 | 46.5 | 45.3 | 54.9 | 42.0 |
| 50 mg/mL | Mean | 405 | 3.73 | 1920 | 1997 | 41.4 | 409 | 25103 |
| | SD | 119 | 1.26 | 532 | 539 | 8.91 | 142 | 13003 |
| | CV % | 29.3 | 33.8 | 27.7 | 27.0 | 21.5 | 34.7 | 51.8 |
| 100 mg/mL | Mean | 200 | 3.54 | 1018 | 1068 | 30 | 828 | 33254 |
| | SD | 88.8 | 1.23 | 443 | 454 | 14.2 | 340 | 20274 |
| | CV % | 44.5 | 34.8 | 43.5 | 42.5 | 47.5 | 41.0 | 61.0 |
| 150 mg/mL | Mean | 180 | 3.21 | 931 | 978 | 28.1 | 894 | 32431 |
| | SD | 87.2 | 1.25 | 381 | 389 | 12.2 | 374 | 12189 |
| | CV % | 458.4 | 38.9 | 40.9 | 39.8 | 43.5 | 41.8 | 37.6 |

TABLE 3
Summary of PK results for 7-OH CBD

| | | Cmax | Tmax | AUC₀₋ₜ | AUC_{0-∞} | |
|---|---|---|---|---|---|---|
| 7-OH-CBD | | (ng/mL) | (h) | (ng · h/mL) | (ng · h/mL) | t1/2 (h) |
| OS | Mean | 153 | 3.26 | 1063 | 1101 | 16.4 |
| | SD | 87.5 | 1.10 | 507 | 511 | 4.20 |
| | CV % | 57.1 | 33.6 | 47.6 | 46.5 | 25.7 |
| 50 mg/mL | Mean | 241 | 3.60 | 1608 | 1649 | 16.6 |
| | SD | 87.1 | 1.10 | 627 | 622 | 3.00 |
| | CV % | 36.1 | 30.6 | 39 | 37.7 | 18 |
| 100 mg/mL | Mean | 162 | 3.15 | 1038 | 1082 | 16.5 |
| | SD | 59.7 | 1.01 | 338 | 330 | 3.29 |
| | CV % | 36.8 | 32 | 32.6 | 32.6 | 19.9 |
| 150 mg/mL | Mean | 168 | 3.11 | 1052 | 1088 | 16.1 |
| | SD | 79.1 | 1.04 | 414 | 421 | 3.89 |
| | CV % | 46.9 | 33.5 | 39.3 | 38.7 | 24.2 |

TABLE 4
Summary of PK results for 7-COOH CBD

| | | Cmax | Tmax | AUC₀₋ₜ | AUC_{0-∞} | |
|---|---|---|---|---|---|---|
| 7-COOH-CBD | | (ng/mL) | (h) | (ng · h/mL) | (ng · h/mL) | t1/2 (h) |
| OS | Mean | 1652 | 4.08 | 47809 | 48946 | 3.10 |
| | SD | 949 | 0.89 | 29314 | 30007 | 3.00 |
| | CV % | 57.4 | 21.9 | 61.3 | 61.3 | 97.4 |
| 50 mg/mL | Mean | 2500 | 4.43 | 67969 | 69514 | 19.6 |
| | SD | 1154 | 0.82 | 37068 | 38164 | 3.27 |
| | CV % | 46.2 | 18.5 | 54.5 | 54.9 | 16.6 |
| 100 mg/mL | Mean | 1694 | 4.36 | 45895 | 46993 | 19.3 |
| | SD | 795 | 0.84 | 23604 | 24310 | 3.31 |
| | CV % | 47.0 | 19.3 | 51.4 | 51.7 | 17.1 |
| 150 mg/mL | Mean | 1829 | 4.01 | 51104 | 52423 | 19.9 |
| | SD | 964 | 0.86 | 29006 | 29873 | 3.80 |
| | CV % | 52.7 | 21.3 | 56.8 | 57.0 | 19.1 |

As can be seen in Table 5 below the ratio of the test formulation to that of the reference formulation (Formulation 1) was much higher for Formulation 2 (50 mg/ml). Formulations 3 and 4 both provided ratios of around 1 meaning that these were effectively bioequivalent to the reference formulation.

TABLE 5
Ratio of test formulations to reference formulation

| | | Ratio of Geo Mean Test:Ref | | |
|---|---|---|---|---|
| | | CBD | 7-OH-CBD | 7-COOH-CBD |
| Cmax | 50 mg/mL | 2.458 | 1.776 | 1.665 |
| (ng/mL) | 100 mg/mL | 1.174 | 1.226 | 1.150 |
| | 150 mg/mL | 1.029 | 1.167 | 1.099 |
| AUC0-t | 50 mg/mL | 2.078 | 1.627 | 1.567 |
| (ng · | 100 mg/mL | 1.068 | 1.083 | 1.079 |
| h/mL) | 150 mg/mL | 0.960 | 1.028 | 1.044 |
| AUC0-inf | 50 mg/mL | 2.061 | 1.607 | 1.553 |
| (ng · | 100 mg/mL | 1.070 | 1.088 | 1.071 |
| h/mL) | 150 mg/mL | 0.961 | 1.023 | 1.038 |

Table 6 details the ratio of CBD metabolites to parent compound CBD for the four different formulations.

TABLE 6
Ratio of CBD metabolites to parent (CBD)

| | | Ratio of Geo Mean Metabolite:Parent | |
|---|---|---|---|
| | | 7-OH-CBD | 7-COOH-CBD |
| Cmax | OS | 0.805 | 8.804 |
| (ng/mL) | 50 mg/mL | 0.582 | 5.967 |
| | 100 mg/mL | 0.841 | 8.625 |
| | 150 mg/mL | 0.913 | 9.406 |
| AUC0-t | OS | 1.043 | 43.224 |
| (ng · | 50 mg/mL | 0.817 | 32.604 |
| h/mL) | 100 mg/mL | 1.057 | 43.652 |
| | 150 mg/mL | 1.116 | 46.973 |
| AUC0-∞ | OS | 1.034 | 42.371 |
| (ng · | 100 mg/mL | 0.806 | 31.923 |
| h/mL) | 100 mg/mL | 1.051 | 42.423 |
| | 150 mg/mL | 1.100 | 45.773 |

These data show that there is a two-fold increase in the bioavailability of CBD for Formulation 2 (50 mg/ml CBD). There was also a difference in the metabolite:parent ratio for this formulation. The ratio of CBD metabolites to the parent compound CBD was lowered in Formulation 2 compared to the other formulations.

### Conclusions:

The data presented in this example demonstrates that the provision of a lower concentration of CBD in an oral formulation was able to produce better bioavailability. Furthermore, these data showed that the lower concentration CBD formulation produced a more beneficial metabolite to parent ratio.

These data are significant as the 7-COOH CBD metabolite of CBD is inactive and as such reducing the amount of formation of this metabolite would suggest that there will be an increased action of the parent compound.

Additional advantages of the lower concentration CBD formulation will be on the undesired liver toxicity associated with CBD. In clinical trials it was found that Epidiolex (plant derived highly purified CBD) could cause liver transaminase (alanine aminotransferase— ALT and/or aspartate aminotransferase—AST) elevations, particularly when used in combination with other anti-epileptic drugs such as clobazam and valproate.

In controlled studies, the incidence of ALT elevations above 3 times the upper limit of normal was 13% in Epidiolex-treated patients compared with 1% in patients on placebo and less than 1% of Epidiolex-treated patients had ALT or AST levels greater than 20 times the upper limit of normal.

### Example 2— Pharmacokinetic Testing of Cbd Formulations in Rat

The pharmacokinetic properties of various oral formulations comprising cannabidiol (CBD) were tested in rat.

The composition of these formulations are described in Table 7 below.

TABLE 7
Composition of formulations

| | CBD | | Dose |
|---|---|---|---|
| Formulation | concentration | | Volume |
| No. | (mg/mL) | Edible oil | (mg/mL) |
| 1 | 50 | Sesame oil | 1.0 |
| 2 | 100 | Sesame oil | 0.5 |
| 3 | 50 | Olive oil | 1.0 |
| 4 | 100 | Olive oil | 0.5 |
| 5 | 200 | Olive oil | 0.25 |
| 6 | 50 | Soybean oil | 1.0 |
| 7 | 100 | Soybean oil | 0.5 |
| 8 | 200 | Soybean oil | 0.25 |
| 9 | 50 | MCT oil | 1.0 |
| 10 | 100 | MCT oil | 0.5 |
| 11 | 200 | MCT oil | 0.25 |
| 12 | 50 | Hemp oil | 1.0 |
| 13 | 100 | Hemp oil | 0.5 |
| 14 | 200 | Hemp oil | 0.25 |

### Protocol Details

The animals were acclimatised for a minimum period of 5 days. The rats were kept in rooms thermostatically maintained within a temperature of 19 to 25° C., with a relative humidity of between 40 and 70%, and exposed to fluorescent light (nominal 12 hours) each day.

An appropriate amount of the required test substance was transferred to a suitable formulation vessel, and an appropriate volume of the required oil vehicle was added to achieve the necessary final concentration, as detailed in Appendix 4.

Each animal received a single oral dose, administered by gavage at a nominal dose level of 50 mg/kg and at a nominal dose volume between 0.25 and 1 mL/kg. The amount of dose formulation administered to each animal was determined from the weight difference between the dosing equipment pre- and post-dose, together with the concentration of the dose formulation (based on the weights of the test substance and the total formulation weight).

Three animals were tested for each formulation.

Following dosing, animals were returned to holding cages. Blood samples (ca. 150 μL) were collected by venepuncture from the jugular vein at each of the following time points:

0.5, 1, 2, 4, 8, 16 and 24 hours post-dose.

Samples were collected into tubes containing K2EDTA anticoagulant and centrifuged (2300 g, 10 minutes, 4° C.) within 30 minutes to produce plasma. Blood cells were discarded. Samples were processed and frozen at <−50° C. within 1 hour of sample collection.

Following the last blood-sampling occasion, animals were killed by euthanasia via overdose of sodium pentobarbitone (death confirmed by cervical dislocation or exsanguination).

### Results

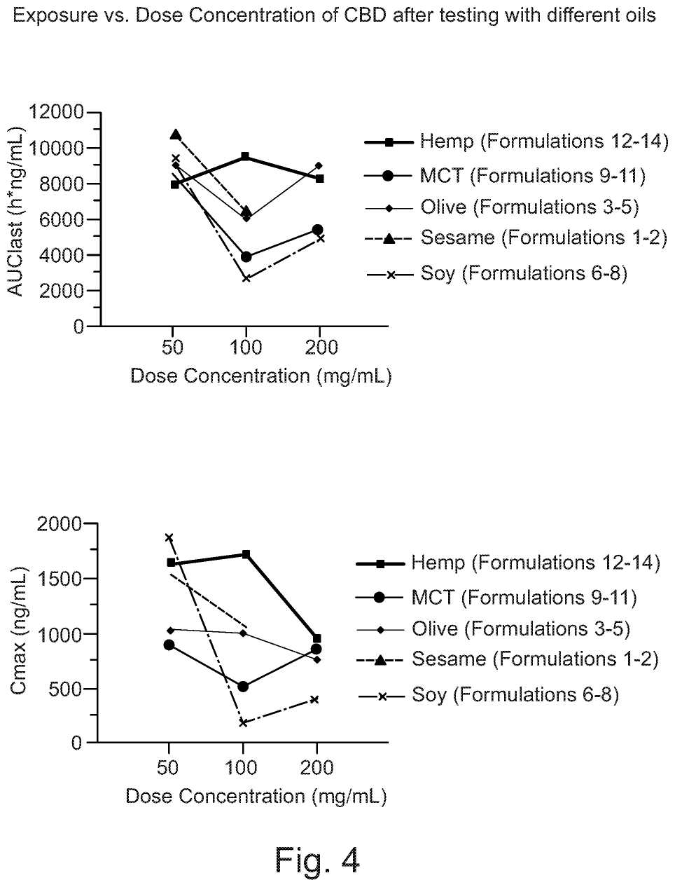

FIG. 4 shows exposure, as assessed by two pharmacokinetic parameters, AUCₗₐₛₜ and Cₘₐₓ, for the different formulations tested. Tables 8 to 10 summarise the mean pharmacokinetic parameters.

### Formulations 1 and 2 (Sesame Oil)

As can be seen in FIG. 4 and Table 8, Formulation 1 produced the higher AUCₗₐₛₜ and Cₘₐₓ values when compared to the other formulation in sesame oil (Formulation 2). This is consistent with the results of Example 1 and is again unexpected as this was the lower concentration of CBD at 50 mg/ml.

As would be expected, Formulation 1 also produced an increased concentration of the two metabolites 7-OH CBD and 7-COOH CBD when compared to the Formulation 2. This is evidenced by Tables 9 and 10.

### Formulations 3, 4 and 5 (Olive Oil)

Similar to Formulations 1 and 2, Formulation 3 produced a higher AUCₗₐₛₜ and Cₘₐₓ values when compared to the higher CBD formulation of Formulation 4 (see FIG. 4). At the highest CBD concentration of 200 mg/ml (Formulation 5), the Cₘₐₓ was surprisingly still lower than at 100 mg/ml. This again provides data to show the increased bioavailability of low CBD concentration formulations.

### Formulations 6, 7 and 8 (Soybean Oil)

Formulation 6 had the highest Cₘₐₓ value out of all the formulations tested as shown by FIG. 4. Compared to Formulations 7 and 8 at higher CBD concentrations, Formulation 6 had higher bioavailability of CBD.

Tables 9 and 10 show that Formulation 6 also produced an increased concentration of the two metabolites 7-OH CBD and 7-COOH CBD when compared to the Formulations 7 and 8.

### Formulations 9, 10 and 11 (MCT Oil)

Formulation 9 had higher AUCₗₐₛₜ and Cₘₐₓ values compared to both Formulations 10 and 11 despite having the lowest concentration of CBD at 50 mg/ml (see FIG. 4 and Table 8).

As would be expected, Formulation 9 also produced an increased concentration of the two metabolites 7-OH CBD and 7-COOH CBD when compared to the Formulations 10 and 11, evidenced by Tables 9 and 10.

### Formulations 12, 13 and 14 (Hemp Oil)

Formulation 13 produced higher AUCₗₐₛₜ and Cₘₐₓ values than Formulations 12 and 14.

TABLE 8
Mean Pharmacokinetic Parameters for CBD in
Rat Plasma following a Single Administration of CBD

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 1550 | 4 | 11500 | 11800 | 4.22 |
| 2 | Mean | 1070 | 4 | 7040 | 7200 | 4.04 |
| 3 | Mean | 1040 | 4 | 9190 | NR | NR |
| 4 | Mean | 996 | 4 | 6500 | 6690 | 4.64 |
| 5 | Mean | 731 | 4 | 9390 | 9720 | 4.33 |
| 6 | Mean | 1870 | 4 | 10700 | NR | NR |
| 7 | Mean | 199 | 2 | 2770 | 3180 | 7.44 |
| 8 | Mean | 403 | 8 | 5020 | NR | NR |
| 9 | Mean | 882 | 8 | 9360 | NR | NR |
| 10 | Mean | 515 | 4 | 4290 | 4410 | 3.98 |
| 11 | Mean | 836 | 4 | 5850 | 5980 | 4.09 |
| 12 | Mean | 1610 | 4 | 8690 | 8880 | 4.28 |
| 13 | Mean | 1700 | 4 | 10500 | 10700 | 3.74 |
| 14 | Mean | 913 | 4 | 8590 | 9030 | 5.12 |

NR Not recorded

TABLE 9
Mean Pharmacokinetic Parameters for 7-OH CBD in Rat
Plasma following a Single Oral Administration of CBD

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 253 | 4.00 | 1610 | 1640 | 3.82 |
| | SD | 109 | 0.00 | 272 | 272 | 0.506 |
| | CV % | 43.1 | 0.00 | 16.8 | 16.6 | 13.2 |
| 2 | Mean | 177 | 4.00 | 1210 | 1220 | 3.33 |
| | SD | 59.7 | 0.00 | 236 | 241 | 0.185 |
| | CV % | 33.8 | 0.00 | 19.5 | 19.7 | 5.57 |
| 3 | Mean | 128 | 10.7 | 1330 | 1810 | 3.85 |
| | SD | 82.2 | 11.5 | 832 | NA | NA |
| | CV % | 64.1 | 108 | 62.4 | NA | NA |
| 4 | Mean | 150 | 4.00 | 1070 | 1100 | 4.20 |
| | SD | 52.4 | 0.00 | 261 | 263 | 0.516 |
| | CV % | 34.8 | 0.00 | 24.3 | 24.0 | 12.3 |
| 5 | Mean | 149 | 4.00 | 1450 | 668 | 3.73 |
| | SD | 83.5 | 3.46 | 1380 | NA | NA |
| | CV % | 56.0 | 86.6 | 95.1 | NA | NA |
| 6 | Mean | 108 | 4.67 | 1070 | 822 | 3.28 |
| | SD | 62.1 | 3.06 | 831 | NA | NA |
| | CV % | 57.4 | 65.5 | 78.0 | NA | NA |
| 7 | Mean | 46.1 | 2.00 | 515 | 532 | 3.70 |
| | SD | 14.7 | 0.00 | 115 | NA | NA |
| | CV % | 32.0 | 0.00 | 22.4 | NA | NA |
| 8 | Mean | 92.1 | 6.00 | 942 | 712 | 3.30 |
| | SD | 29.6 | 3.46 | 382 | NA | NA |
| | CV % | 32.2 | 57.7 | 40.5 | NA | NA |
| 9 | Mean | 179 | 5.33 | 1570 | 1590 | 4.34 |
| | SD | 21.7 | 2.31 | 115 | NA | NA |
| | CV % | 12.1 | 43.3 | 7.30 | NA | NA |
| 10 | Mean | 129 | 3.00 | 925 | 1240 | 3.24 |
| | SD | 99.1 | 1.73 | 623 | NA | NA |
| | CV % | 77.0 | 57.7 | 67.3 | NA | NA |
| 11 | Mean | 175 | 3.33 | 1280 | 1190 | 3.62 |
| | SD | 29.4 | 1.15 | 232 | NA | NA |
| | CV % | 16.8 | 34.6 | 18.1 | NA | NA |
| 12 | Mean | 277 | 4.00 | 1480 | 1490 | 3.39 |
| | SD | 184 | 0.00 | 376 | 379 | 0.506 |
| | CV % | 66.5 | 0.00 | 25.4 | 25.4 | 14.9 |
| 13 | Mean | 234 | 4.00 | 1570 | 1590 | 3.32 |
| | SD | 61.8 | 0.00 | 535 | 540 | 0.179 |
| | CV % | 26.4 | 0.00 | 34.1 | 34.0 | 5.38 |
| 14 | Mean | 188 | 5.33 | 1600 | 1370 | 3.73 |
| | SD | 90.9 | 2.31 | 554 | NA | NA |
| | CV % | 48.3 | 43.3 | 34.7 | NA | NA |

NA Not applicable

TABLE 10
Mean Pharmacokinetic Parameters for 7-COOH CBD in Rat
Plasma following a Single Oral Administration of CBD

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 987 | 5.33 | 8480 | 9830 | 3.35 |
| | SD | 373 | 2.31 | 3390 | NA | NA |
| | CV % | 37.8 | 43.3 | 39.9 | NA | NA |
| 2 | Mean | 828 | 4.00 | 6130 | 6180 | 3.06 |
| | SD | 151 | 0.00 | 1450 | 1460 | 0.155 |
| | CV % | 18.2 | 0.00 | 23.6 | 23.6 | 5.06 |
| 3 | Mean | 1050 | 16.0 | 11300 | NA | NA |
| | SD | 837 | 8.00 | 10800 | NA | NA |
| | CV % | 79.7 | 50.0 | 95.2 | NA | NA |
| 4 | Mean | 854 | 4.00 | 7080 | 10200 | 4.48 |
| | SD | 705 | 0.00 | 5740 | NA | NA |
| | CV % | 82.5 | 0.00 | 81.1 | NA | NA |
| 5 | Mean | 1110 | 5.33 | 12400 | 3810 | 3.23 |
| | SD | 1180 | 2.31 | 15800 | NA | NA |
| | CV % | 106 | 43.3 | 128 | NA | NA |
| 6 | Mean | 494 | 9.33 | 5680 | 4430 | 2.62 |
| | SD | 415 | 6.11 | 4710 | NA | NA |
| | CV % | 83.9 | 65.5 | 82.9 | NA | NA |
| 7 | Mean | 129 | 8.67 | 1620 | NA | NA |
| | SD | 82.2 | 7.02 | 1010 | NA | NA |
| | CV % | 63.8 | 81.0 | 62.3 | NA | NA |
| 8 | Mean | 386 | 10.7 | 5190 | NA | NA |
| | SD | 209 | 4.62 | 3820 | NA | NA |
| | CV % | 54.0 | 43.3 | 73.5 | NA | NA |
| 9 | Mean | 1560 | 6.67 | 18700 | 24700 | 7.28 |
| | SD | 170 | 2.31 | 2640 | NA | NA |
| | CV % | 10.9 | 34.6 | 14.1 | NA | NA |
| 10 | Mean | 918 | 5.33 | 10100 | 2830 | 3.21 |
| | SD | 1100 | 2.31 | 12700 | NA | NA |
| | CV % | 120 | 43.3 | 126 | NA | NA |
| 11 | Mean | 1180 | 5.33 | 12500 | 14800 | 4.37 |
| | SD | 696 | 2.31 | 4960 | NA | NA |
| | CV % | 59.2 | 43.3 | 39.7 | NA | NA |
| 12 | Mean | 787 | 4.00 | 5030 | 6780 | 2.70 |
| | SD | 892 | 0.00 | 4920 | NA | NA |
| | CV % | 113 | 0.00 | 97.8 | NA | NA |
| 13 | Mean | 1350 | 4.00 | 11500 | 11600 | 2.84 |
| | SD | 635 | 0.00 | 5800 | 5860 | 0.220 |
| | CV % | 47.2 | 0.00 | 50.4 | 50.5 | 7.76 |
| 14 | Mean | 711 | 9.33 | 8960 | NA | NA |
| | SD | 495 | 6.11 | 7960 | NA | NA |
| | CV % | 69.6 | 65.5 | 88.9 | NA | NA |

Tables 11.1 to 11.5 below details the ratio of CBD metabolites to parent compound CBD for the different formulations.

TABLE 11.1
Ratio of Metabolite to Parent (Sesame oil)

| | | Ratio of Geo Mean | |
|---|---|---|---|
| | Formulation | Metabolite:Parent | |
| | No. | 7-OH-CBD | 7-COOH-CBD |
| Cₘₐₓ/D | 1 | 0.161 | 0.627 |
| (ng/mL) | 2 | 0.165 | 0.765 |
| AUC₀₋₂₄/D | 1 | 0.141 | 0.742 |
| (ng · h/mL) | 2 | 0.171 | 0.865 |

These data show that there is a difference in the metabolite:parent ratio for these two formulations. The ratio of metabolite to parent was lowered in Formulation 1 compared to Formulation 2.

TABLE 11.2
Ratio of Metabolite to Parent (Olive oil)

| | | Ratio of Geo Mean | |
|---|---|---|---|
| | Formulation | Metabolite:Parent | |
| | No. | 7-OH-CBD | 7-COOH-CBD |
| Cₘₐₓ/D | 3 | 0.138 | 1.14 |
| (ng/mL) | 4 | 0.150 | 0.852 |
| | 5 | 0.192 | 1.24 |
| AUC₀₋₂₄/D | 3 | 0.164 | 1.40 |
| (ng · h/mL) | 4 | 0.164 | 1.08 |
| | 5 | 0.170 | 1.31 |

These data show that there is a difference in the metabolite:parent ratio for these three formulations. The 7-OH CBD:CBD ratio was lowered in Formulation 3 compared to Formulation 5.

TABLE 11.3
Ratio of Metabolite to Parent (Soybean oil)

| | | Ratio of Geo Mean | |
|---|---|---|---|
| | Formulation | Metabolite:Parent | |
| | No. | 7-OH-CBD | 7-COOH-CBD |
| Cₘₐₓ/D | 6 | 0.104 | 0.476 |
| (ng/mL) | 7 | 0.229 | 0.633 |
| | 8 | 0.231 | 0.958 |
| AUC₀₋₂₄/D | 6 | 0.133 | 0.709 |
| (ng · h/mL) | 7 | 0.176 | 0.548 |
| | 8 | 0.190 | 1.02 |

The metabolite to parent ratio was considerably lowered in Formulation 6 compared to Formulations 7 and 8.

TABLE 11.4
Ratio of Metabolite to Parent (MCT oil)

| | | Ratio of Geo Mean | |
|---|---|---|---|
| | Formulation | Metabolite:Parent | |
| | No. | 7-OH-CBD | 7-COOH-CBD |
| Cₘₐₓ/D | 9 | 0.180 | 1.58 |
| (ng/mL) | 10 | 0.251 | 1.84 |
| | 11 | 0.208 | 1.43 |
| AUC₀₋₂₄/D | 9 | 0.168 | 2.00 |
| (ng · h/mL) | 10 | 0.218 | 2.46 |
| | 11 | 0.204 | 2.06 |

The 7-OH CBD:CBD ratio was lowered in Formulation 9 compared to Formulations 10 and 11.

TABLE 11.5
Ratio of Metabolite to Parent (Hemp oil)

| | | Ratio of Geo Mean | |
|---|---|---|---|
| | Formulation | Metabolite:Parent | |
| | No. | 7-OH-CBD | 7-COOH-CBD |
| Cₘₐₓ/D | 12 | 0.172 | 0.488 |
| (ng/mL) | 13 | 0.136 | 0.776 |
| | 14 | 0.175 | 0.673 |
| AUC₀₋₂₄/D | 12 | 0.170 | 0.581 |
| (ng · h/mL) | 13 | 0.148 | 1.08 |
| | 14 | 0.188 | 1.06 |

Both 7-OH CBD:CBD and 7-COOH CBD:CBD ratios were lowered in Formulation 12 compared to Formulation 14.

### Conclusions:

Exposure, as assessed by CBD and metabolite Cₘₐₓ and AUC values, was generally observed to be higher in 50 mg/mL dosing concentration compared to 100 and 200 mg/mL for the different vehicles.

The data presented in this example further demonstrates that the provision of a lower concentration of CBD was able to produce better bioavailability in the different oil formulations tested including sesame, olive, soybean, MCT and hemp oil. This is in line with the results of the low CBD concentration formulation in sesame oil of Example 1 and reaffirms its conclusions.

Furthermore, these data showed that the lower concentration CBD formulation produced a more beneficial metabolite to parent ratio.

As mentioned previously, these data are significant as the 7-COOH CBD metabolite of CBD is inactive and as such reducing the amount of formation of this metabolite would suggest that there will be an increased action of the parent compound.

### Example 3— Pharmacokinetic Testing of Cbdv Formulations in Rat

The pharmacokinetic properties of various oral formulations comprising cannabidivarin (CBDV) were tested in rat.

The composition of these formulations are described in Table 12 below.

TABLE 12
Composition of formulations

| | CBDV | | Dose |
|---|---|---|---|
| Formulation | concentration | | Volume |
| No. | (mg/ml) | Edible oil | (mg/mL) |
| 1 | 25 | Sesame oil | 2.0 |
| 2 | 50 | Sesame oil | 1.0 |
| 3 | 75 | Sesame oil | 0.667 |

### Protocol Details

These formulations were tested in rat as per the protocol described in Example 2.

### Results

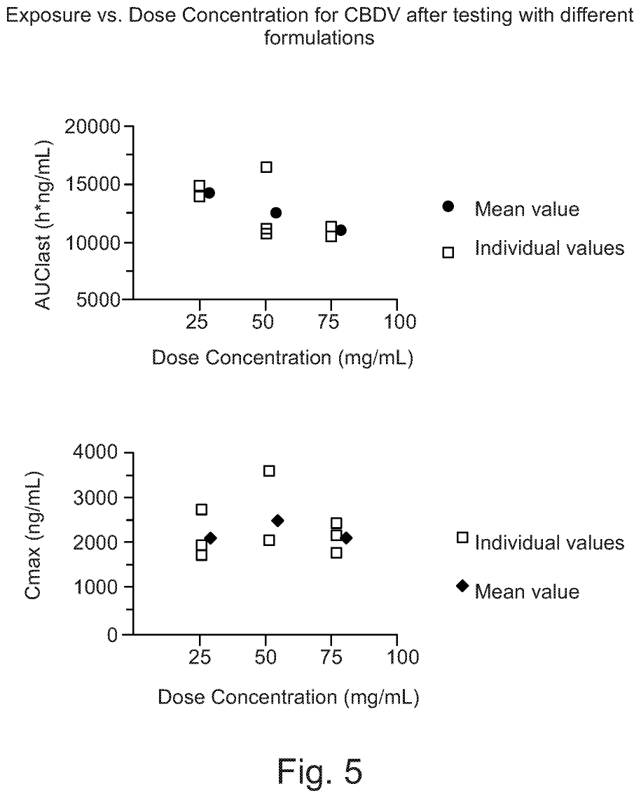

FIG. 5 shows two pharmacokinetic parameters, AUCₗₐₛₜ and Cₘₐₓ, for the different formulations tested. Tables 13 to 15 summarise the mean pharmacokinetic parameters.

As can be seen in FIG. 5, Formulation 1 produced the highest AUCₗₐₛₜ value whilst Formulation 2 produced the highest Cₘₐₓ value out of the three formulations. Thus, the highest concentration of CBDV at 75 mg/ml (Formulation 3) did not produce the highest values as expected. This finding is consistent with the results of Examples 1 and 2 whereby the lower cannabinoid concentration formulations produced better bioavailability results.

As would be expected, Formulations 1 and 2 also produced increased concentrations of the two metabolites 7-OH CBD and 7-COOH CBDV when compared to Formulation 3. This is evidenced by Tables 14 and 15.

TABLE 13
Mean Pharmacokinetic Parameters for CBDV in Rat
Plasma following a Single Oral Administration of CBDV

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 2140 | 4.00 | 15600 | 16100 | 4.06 |
| | SD | 520 | 0.00 | 542 | 611 | 0.429 |
| | CV % | 24.3 | 0.00 | 3.47 | 3.81 | 10.6 |
| 2 | Mean | 2580 | 3.33 | 14400 | 14700 | 3.93 |
| | SD | 875 | 1.15 | 3580 | 3550 | 0.366 |
| | CV % | 33.9 | 34.6 | 24.8 | 24.1 | 9.30 |
| 3 | Mean | 2130 | 4.00 | 12200 | 12600 | 5.05 |
| | SD | 317 | 0.00 | 470 | 440 | 0.594 |
| | CV % | 14.9 | 0.00 | 3.85 | 3.49 | 11.8 |

TABLE 14
Mean Pharmacokinetic Parameters for 7-OH CBDV in Rat
Plasma following a Single Oral Administration of CBDV

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 551 | 3.33 | 6810 | 7100 | 4.68 |
| | SD | 109 | 1.15 | 862 | 773 | 0.688 |
| | CV % | 19.8 | 34.6 | 12.7 | 10.9 | 14.7 |
| 2 | Mean | 879 | 2.33 | 7210 | 7440 | 4.42 |
| | SD | 262 | 1.53 | 1300 | 1150 | 1.14 |
| | CV % | 29.8 | 65.5 | 18.0 | 15.5 | 25.8 |
| 3 | Mean | 714 | 2.67 | 5650 | 5910 | 5.06 |
| | SD | 39.5 | 1.15 | 634 | 603 | 0.462 |
| | CV % | 5.53 | 43.3 | 11.2 | 10.2 | 9.13 |

TABLE 15
Mean Pharmacokinetic Parameters for 7-COOH CBDV in Rat
Plasma following a Single Oral Administration of CBDV

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 8040 | 10.7 | 126000 | NA | NA |
| | SD | 4890 | 4.62 | 78500 | NA | NA |
| | CV % | 60.7 | 43.3 | 62.5 | NA | NA |
| 2 | Mean | 7300 | 10.7 | 104000 | NA | NA |
| | SD | 1900 | 4.62 | 33500 | NA | NA |
| | CV % | 26.0 | 43.3 | 32.3 | NA | NA |
| 3 | Mean | 1640 | 5.33 | 22300 | 12100 | 4.81 |
| | SD | 1050 | 2.31 | 18800 | NA | NA |
| | CV % | 64.0 | 43.3 | 84.1 | NA | NA |

### Conclusions:

Exposure, as assessed by CBDV and metabolite Cₘₐₓ and AUC values, increased as the dosing concentration decreased from 75 to 25 mg/mL for the formulations in sesame oil.

The data presented in this example demonstrates that the provision of a lower concentration of CBDV was able to produce better bioavailability in the different formulations. This is in line with the results of Examples 1 and 2, providing further evidence that formulations with a lower cannabinoid concentration can lead to unexpected, beneficial effects.

### Example 4— Pharmacokinetic Testing of Cbd-C4 Formulations in Rat

The pharmacokinetic properties of various oral formulations comprising cannabidiol-C4 (CBD-C4) were tested.

The composition of these formulations are described in Table 16 below.

TABLE 16
Composition of formulations

| | CBD-C4 | | Dose |
|---|---|---|---|
| Formulation | concentration | | Volume |
| No. | (mg/ml) | Edible oil | (mg/mL) |
| 1 | 50 | Sesame oil | 1.0 |
| 2 | 100 | Sesame oil | 0.5 |
| 3 | 200 | Sesame oil | 0.25 |
| 4 | 50 | Olive oil | 1.0 |
| 5 | 100 | Olive oil | 0.5 |
| 6 | 200 | Olive oil | 0.25 |
| 7 | 50 | Soy oil | 1.0 |
| 8 | 100 | Soy oil | 0.5 |
| 9 | 200 | Soy oil | 0.25 |
| 10 | 50 | MCT oil | 1.0 |
| 11 | 100 | MCT oil | 0.5 |
| 12 | 200 | MCT oil | 0.25 |
| 13 | 50 | Hemp oil | 1.0 |
| 14 | 100 | Hemp oil | 0.5 |
| 15 | 200 | Hemp oil | 0.25 |

### Protocol Details

These formulations were tested in rat as per the protocol described in Example 2.

### Results

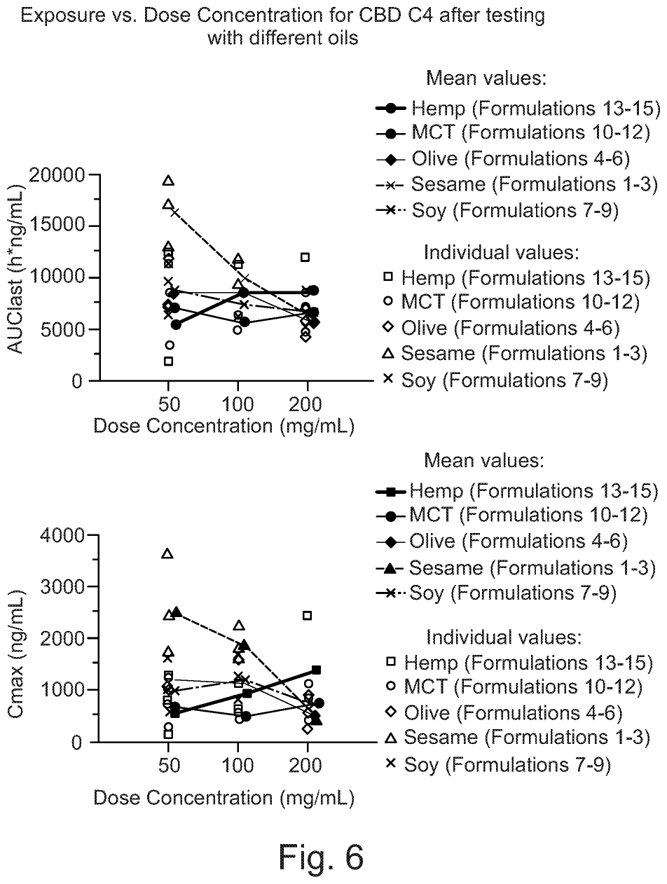

FIG. 6 shows two pharmacokinetic parameters, AUCₗₐₛₜ and Cₘₐₓ, for the different formulations tested. Tables 17 to 19 summarise the mean pharmacokinetic parameters.

### Formulations 1, 2 and 3 (Sesame Oil)

As can be seen in FIG. 6, Formulation 1 produced the a higher AUCₗₐₛₜ and Cₘₐₓ values when compared to the other two formulations in sesame oil (Formulations 2 and 3). This is consistent with the results of Examples 1, 2 and 3 whereby the lower cannabinoid concentration formulations gave better bioavailability.

### Formulations 4, 5 and 6 (Olive Oil)

Similar to Formulations 1, the lower concentration Formulation 3 produced better bioavailability results when compared to the higher CBD-C4 formulations of Formulations 4 and 5 (see FIG. 6).

### Formulations 7, 8 and 9 (Soybean Oil)

Formulations in soybean oil displayed a similar pattern whereby the 200 mg/ml CBD-C4 formulation, Formulation 9, produced the lowest AUCₗₐₛₜ and Cₘₐₓ values out of the three formulations. Compared to Formulations 7 and 8 at higher CBD concentrations, Formulation 6 had higher bioavailability of CBD.

Tables 18 and 19 show that Formulation 7 also produced an increased concentration of the two metabolites 7-OH CBD and 7-COOH CBD when compared to the Formulations 8 and 9.

### Formulations 10, 11 and 12 (MCT Oil)

Medium dose Formulation 11 produced lower AUCₗₐₛₜ and Cₘₐₓ values compared to Formulations 10 and 12 (see FIG. 6).

### Formulations 13, 14 and 15 (Hemp Oil)

Formulation 15 produced higher AUCₗₐₛₜ and Cₘₐₓ values compared to Formulations 13 and 14.

TABLE 17
Mean Pharmacokinetic Parameters for CBD-C4 in Rat
Plasma following a Single Oral Administration of CBD-C4

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 2610 | 4.00 | 17800 | 18800 | 5.28 |
| | SD | 954 | 0.00 | 3450 | 3680 | 1.03 |
| | CV % | 36.5 | 0.00 | 19.3 | 19.6 | 19.4 |
| 2 | Mean | 1910 | 4.00 | 11100 | 11600 | 5.21 |
| | SD | 328 | 0.00 | 1640 | 1660 | 0.141 |
| | CV % | 17.2 | 0.00 | 14.7 | 14.3 | 2.70 |
| 3 | Mean | 523 | 10.7 | 6400 | NA | NA |
| | SD | 265 | 4.62 | 2290 | NA | NA |
| | CV % | 50.7 | 43.3 | 35.8 | NA | NA |
| 4 | Mean | 1270 | 2.67 | 9070 | 10500 | 5.74 |
| | SD | 353 | 1.15 | 2850 | NA | NA |
| | CV % | 27.8 | 43.3 | 31.5 | NA | NA |
| 5 | Mean | 1190 | 4.67 | 9180 | 9230 | 5.38 |
| | SD | 354 | 3.06 | 1240 | NA | NA |
| | CV % | 29.8 | 65.5 | 13.5 | NA | NA |
| 6 | Mean | 601 | 6.00 | 5880 | 7770 | 5.60 |
| | SD | 334 | 3.46 | 1550 | NA | NA |
| | CV % | 55.5 | 57.7 | 26.3 | NA | NA |
| 7 | Mean | 1100 | 5.33 | 9700 | 9840 | 4.42 |
| | SD | 494 | 2.31 | 2770 | NA | NA |
| | CV % | 45.1 | 43.3 | 28.5 | NA | NA |
| 8 | Mean | 1270 | 4.00 | 8100 | 8390 | 4.57 |
| | SD | 430 | 0.00 | 1530 | 1430 | 0.648 |
| | CV % | 34.0 | 0.00 | 18.9 | 17.0 | 14.2 |
| 9 | Mean | 835 | 8.00 | 7560 | NA | NA |
| | SD | 465 | 0.00 | 2680 | NA | NA |
| | CV % | 55.7 | 0.00 | 35.5 | NA | NA |
| 10 | Mean | 776 | 6.67 | 8360 | 12900 | 3.85 |
| | SD | 474 | 2.31 | 4520 | NA | NA |
| | CV % | 61.1 | 34.6 | 54.0 | NA | NA |
| 11 | Mean | 522 | 5.33 | 5940 | 5910 | 4.88 |
| | SD | 51.0 | 2.31 | 751 | NA | NA |
| | CV % | 9.77 | 43.3 | 12.6 | NA | NA |
| 12 | Mean | 834 | 5.33 | 7320 | 8910 | 4.84 |
| | SD | 356 | 2.31 | 2210 | NA | NA |
| | CV % | 42.7 | 43.3 | 30.1 | NA | NA |
| 13 | Mean | 752 | 4.00 | 7480 | 5070 | 5.37 |
| | SD | 575 | 0.00 | 5590 | NA | NA |
| | CV % | 76.4 | 0.00 | 74.7 | NA | NA |
| 14 | Mean | 988 | 5.33 | 9540 | 8820 | 5.87 |
| | SD | 274 | 2.31 | 3080 | NA | NA |
| | CV % | 27.7 | 43.3 | 32.3 | NA | NA |
| 15 | Mean | 1510 | 4.00 | 9700 | 10100 | 4.86 |
| | SD | 808 | 0.00 | 2940 | 3020 | 0.426 |
| | CV % | 53.4 | 0.00 | 30.3 | 30.0 | 8.78 |

NA Not applicable

TABLE 18
Mean Pharmacokinetic Parameters for 7-OH CBD-C4 in Rat
Plasma following a Single Oral Administration of CBD-C4

| Formulation | | Cₘₐₓ | | AUC₀₋ₜ | AUC_{0-inf} | |
|---|---|---|---|---|---|---|
| No. | | (ng/mL) | Tₘₐₓ (h) | (ng · h/mL) | (ng · h/mL) | t_{1/2} (h) |
| 1 | Mean | 534 | 4.00 | 4740 | 5010 | 5.18 |
| | SD | 26.6 | 0.00 | 837 | 786 | 0.766 |
| | CV % | 4.98 | 0.00 | 17.6 | 15.7 | 14.8 |
| 2 | Mean | 632 | 4.00 | 4680 | 4800 | 4.19 |
| | SD | 217 | 0.00 | 791 | 768 | 0.403 |
| | CV % | 34.3 | 0.00 | 16.9 | 16.0 | 9.63 |
| 3 | Mean | 280 | 6.67 | 3240 | 4850 | 4.45 |
| | SD | 167 | 2.31 | 1230 | NA | NA |
| | CV % | 59.8 | 34.6 | 37.8 | NA | NA |
| 4 | Mean | 578 | 2.67 | 3990 | 4260 | 5.33 |
| | SD | 83.5 | 1.15 | 778 | NA | NA |
| | CV % | 14.4 | 43.3 | 19.5 | NA | NA |
| 5 | Mean | 760 | 3.33 | 4820 | 5120 | 5.25 |
| | SD | 321 | 1.15 | 484 | 464 | 1.03 |
| | CV % | 42.3 | 34.6 | 10.0 | 9.07 | 19.7 |
| 6 | Mean | 377 | 6.00 | 3240 | 4190 | 5.17 |
| | SD | 247 | 3.46 | 693 | NA | NA |
| | CV % | 65.4 | 57.7 | 21.4 | NA | NA |
| 7 | Mean | 513 | 4.00 | 4350 | 4590 | 5.01 |
| | SD | 249 | 0.00 | 1350 | 1400 | 1.57 |
| | CV % | 48.5 | 0.00 | 31.0 | 30.4 | 31.3 |
| 8 | Mean | 457 | 4.00 | 3210 | 3310 | 4.37 |
| | SD | 125 | 0.00 | 545 | 558 | 0.442 |
| | CV % | 27.4 | 0.00 | 16.9 | 16.9 | 10.1 |
| 9 | Mean | 331 | 8.00 | 3410 | NA | NA |
| | SD | 92.6 | 0.00 | 584 | NA | NA |
| | CV % | 27.9 | 0.00 | 17.1 | NA | NA |
| 10 | Mean | 384 | 3.33 | 3870 | 3970 | 3.99 |
| | SD | 173 | 1.15 | 1640 | 1640 | 0.790 |
| | CV % | 45.0 | 34.6 | 42.3 | 41.3 | 19.8 |
| 11 | Mean | 439 | 3.33 | 3820 | 3960 | 4.42 |
| | SD | 216 | 1.15 | 934 | 978 | 0.552 |
| | CV % | 49.2 | 34.6 | 24.4 | 24.7 | 12.5 |
| 12 | Mean | 374 | 3.33 | 3460 | 3590 | 4.44 |
| | SD | 114 | 1.15 | 785 | 859 | 0.736 |
| | CV % | 30.6 | 34.6 | 22.7 | 24.0 | 16.6 |
| 13 | Mean | 388 | 2.67 | 3990 | 4250 | 4.78 |
| | SD | 211 | 1.15 | 2550 | 2840 | 1.65 |
| | CV % | 54.2 | 43.3 | 63.8 | 66.9 | 34.6 |
| 14 | Mean | 508 | 3.33 | 4520 | 4760 | 4.99 |
| | SD | 216 | 1.15 | 1390 | 1480 | 1.50 |
| | CV % | 42.6 | 34.6 | 30.7 | 31.0 | 30.1 |
| 15 | Mean | 493 | 3.33 | 3800 | 3930 | 4.62 |
| | SD | 124 | 1.15 | 702 | 747 | 0.403 |
| | CV % | 25.2 | 34.6 | 18.5 | 19.0 | 8.74 |

TABLE 19
Mean Pharmacokinetic Parameters for 7-COOH CBD-C4 in Rat
Plasma following a Single Oral Administration of CBD-C4

| | | Cₘₐₓ | | AUC₀₋ₜ | AUC_{0-inf} | |
|---|---|---|---|---|---|---|
| Formulation No. | | (ng/mL) | Tₘₐₓ (h) | (ng · h/mL) | (ng · h/mL) | t_{1/2} (h) |
| 1 | Mean | 2350 | 8.00 | 32600 | NA | NA |
| | SD | 834 | 0.00 | 14500 | NA | NA |
| | CV % | 35.5 | 0.00 | 44.3 | NA | NA |
| 2 | Mean | 3750 | 8.00 | 52600 | NA | NA |
| | SD | 2410 | 0.00 | 38300 | NA | NA |
| | CV % | 64.3 | 0.00 | 72.9 | NA | NA |
| 3 | Mean | 2270 | 10.7 | 35400 | NA | NA |
| | SD | 1390 | 4.62 | 24000 | NA | NA |
| | CV % | 61.3 | 43.3 | 67.9 | NA | NA |
| 4 | Mean | 2820 | 6.67 | 53300 | NA | NA |
| | SD | 2150 | 2.31 | 47000 | NA | NA |
| | CV % | 76.1 | 34.6 | 88.3 | NA | NA |
| 5 | Mean | 4620 | 8.00 | 78300 | NA | NA |
| | SD | 3080 | 0.00 | 56900 | NA | NA |
| | CV % | 66.6 | 0.00 | 72.7 | NA | NA |
| 6 | Mean | 2120 | 13.3 | 33400 | NA | NA |
| | SD | 972 | 9.24 | 17000 | NA | NA |
| | CV % | 45.9 | 69.3 | 51.0 | NA | NA |
| 7 | Mean | 5380 | 10.7 | 87700 | NA | NA |
| | SD | 1980 | 4.62 | 34300 | NA | NA |
| | CV % | 36.7 | 43.3 | 39.1 | NA | NA |
| 8 | Mean | 3760 | 8.00 | 50200 | NA | NA |
| | SD | 910 | 0.00 | 13800 | NA | NA |
| | CV % | 24.2 | 0.00 | 27.5 | NA | NA |
| 9 | Mean | 1180 | 10.7 | 16200 | NA | NA |
| | SD | 292 | 4.62 | 3740 | NA | NA |
| | CV % | 24.8 | 43.3 | 23.1 | NA | NA |
| 10 | Mean | 4310 | 8.00 | 61400 | NA | NA |
| | SD | 2680 | 0.00 | 36400 | NA | NA |
| | CV % | 62.1 | 0.00 | 59.3 | NA | NA |
| 11 | Mean | 4780 | 8.00 | 79700 | NA | NA |
| | SD | 2360 | 0.00 | 44400 | NA | NA |
| | CV % | 49.4 | 0.00 | 55.7 | NA | NA |
| 12 | Mean | 3060 | 8.00 | 41400 | NA | NA |
| | SD | 1510 | 0.00 | 21200 | NA | NA |
| | CV % | 49.3 | 0.00 | 51.2 | NA | NA |
| 13 | Mean | 2390 | 6.67 | 36700 | 5240 | 4.41 |
| | SD | 1720 | 2.31 | 28900 | NA | NA |
| | CV % | 72.1 | 34.6 | 78.8 | NA | NA |
| 14 | Mean | 1980 | 6.67 | 31600 | 15200 | 5.17 |
| | SD | 688 | 2.31 | 17500 | NA | NA |
| | CV % | 34.7 | 34.6 | 55.4 | NA | NA |
| 15 | Mean | 2330 | 6.67 | 36600 | 12200 | 6.63 |
| | SD | 2400 | 2.31 | 43000 | NA | NA |
| | CV % | 103 | 34.6 | 118 | NA | NA |

Tables 20.1 to 20.2 below detail the ratio of CBD-C4 metabolites to parent compound CBD-C4 for the different formulations.

TABLE 20.1
Ratio of Metabolite to Parent (Sesame oil)

| | | Ratio of Geo Mean | |
|---|---|---|---|
| | Formulation | Metabolite:Parent | |
| | No. | 7-OH-CBD-C4 | 7-COOH-CBD-C4 |
| Cₘₐₓ/D | 1 | 0.205 | 0.906 |
| (ng/mL) | 2 | 0.331 | 1.96 |
| | 3 | 0.531 | 4.38 |
| AUC₀₋₂₄/D | 1 | 0.266 | 1.84 |
| (ng · h/mL) | 2 | 0.421 | 4.73 |
| | 3 | 0.507 | 5.52 |

These data show that there is a difference in the metabolite:parent ratio for these three formulations. The ratio of metabolite to parent was lowered in Formulation 1 compared to Formulations 2 and 3.

TABLE 20.2
Ratio of Metabolite to Parent (Olive oil)

| | | Ratio of Geo Mean | |
|---|---|---|---|
| | Formulation | Metabolite:Parent | |
| | No. | 7-OH-CBD-C4 | 7-COOH-CBD-C4 |
| Cₘₐₓ/D | 4 | 0.454 | 2.23 |
| (ng/mL) | 5 | 0.648 | 3.95 |
| | 6 | 0.635 | 3.60 |
| AUC₀₋₂₄/D | 4 | 0.441 | 5.92 |
| (ng · h/mL) | 5 | 0.529 | 8.74 |
| | 6 | 0.556 | 5.64 |

The metabolite to parent ratio was lowered in Formulation 4 compared to Formulations 5 and 6.

### Conclusions:

Exposure, as assessed by CBD-C4 and metabolite Cₘₐₓ and AUC values, increased as the dosing concentration decreased from 200 to 50 mg/mL for the different vehicles.

The data presented in this example demonstrates that a lower concentration of CBD-C4 was able to produce better bioavailability in the different oil formulations tested.

Furthermore, these data showed that the lower concentration CBD-C4 formulation produced a more beneficial metabolite to parent ratio.

Thus, the data additionally verifies the results of the previous examples whereby the provision of a lower concentration of cannabinoid in an oral formulation leads to surprising and unexpected effects of increased bioavailability.

1. A cannabinoid containing oral solution which comprises: a cannabinoid and a lipid solvent, characterised in that the cannabinoid is present in a concentration of from 25 to 75 mg/ml.

2. A cannabinoid containing oral solution according to claim 1, characterised in that the Cₘₐₓ produced in a human is greater than 250 ng/ml.

3. A cannabinoid containing oral solution according to claim 1, wherein the AUC₀₋ₜ produced in a human is greater than 1250 ng.h/ml.

4. A cannabinoid containing oral solution according to any of the preceding claims, wherein the cannabinoid is selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV); tetrahydrocannabivarinic acid (THCVA); cannabidiol-C1 (CBD-C1); cannabidiol-C4 (CBD-C4); and cannabidiol-C6 (CBD-C6).

5. A cannabinoid containing oral solution according to claim 4, wherein the cannabinoid is cannabidiol (CBD).

6. A cannabinoid containing oral solution according to any of the preceding claims, wherein the cannabinoid is present at a concentration of approximately 50 mg/ml.

7. A cannabinoid containing oral solution according to any of the preceding claims, wherein the lipid solvent is an edible oil.

8. A cannabinoid containing oral solution according to claim 4, wherein the edible oil is selected from: coconut oil; corn oil; cottonseed oil; hemp oil; olive oil; palm oil; peanut oil; rapeseed/canola oil; safflower oil; sesame oil; soybean oil; short chain triglyceride; medium chain triglyceride; long chain triglyceride and sunflower oil.

9. A cannabinoid containing oral solution according to claim 8, wherein the cannabinoid is cannabidiol (CBD) and the edible oil is sesame oil.

10. A cannabinoid containing oral solution according to claim 8, wherein the cannabinoid is cannabidiol (CBD) and the edible oil is soybean oil.

11. A cannabinoid containing oral solution according to claim 8, wherein the cannabinoid is cannabidiol (CBD) and the edible oil is olive oil.

12. A cannabinoid containing oral solution according to claim 8, wherein the cannabinoid is cannabidiol (CBD) and the edible oil is medium chain triglyceride.

13. A cannabinoid containing oral solution according to claim 8, wherein the cannabinoid is cannabidivarin (CBDV) and the edible oil is sesame oil.

14. A cannabinoid containing oral solution according to claim 8, wherein the cannabinoid is cannabidiol-C4 (CBD-C4) and the edible oil is sesame oil.

15. A cannabinoid containing oral solution according to claim 8, wherein the cannabinoid is cannabidiol-C4 (CBD-C4) and the edible oil is soybean oil.

16. A cannabinoid containing oral solution according to claim 8, wherein the cannabinoid is cannabidiol-C4 (CBD-C4) and the edible oil is olive oil.

17. A cannabinoid containing oral solution according to claim 8, wherein the cannabinoid is cannabidiol-C4 (CBD-C4) and the edible oil is medium chain triglyceride.

18. A cannabinoid containing oral solution according to any of the preceding claims, which further comprises ethanol.

19. A cannabinoid containing oral solution according to claim 18, wherein the ethanol is present at less than 10% w/v.

20. A cannabinoid containing oral solution according to claim 18, wherein the ethanol is present at less than 1% w/v.

21. A cannabinoid containing oral solution according to any of the preceding claims, for use in the treatment of a disease or disorder selected from the group consisting of: epilepsy and syndromes associated therewith, Dravet Syndrome, Lennox Gastaut Syndrome, myocolonic seizures, juvenile mycolonic epilepsy, refractory epilepsy, schizophrenia, juvenile spasms, West syndrome, infantile spasms, refractory infantile spasms, tuberous sclerosis complex, brain tumors, neuropathic pain, cannabis use disorder, post-traumatic stress disorder, anxiety, early psychosis, Alzheimer's Disease, and autism.
